(19) United States
(12) Patent Application Publication
(10) Pub. No.: US 2007/0031406 A1
(43) Pub. Date: Feb. 8, 2007

(54) Anti-thymocyte antiserum and use thereof to trigger b cell apoptosis

(76) Inventors: Martin S. Zand, Rochester, NY (US); Jainulabdeen J. Ifthikharuddin, Rochester, NY (US); Jane Liesveld, Rochester, NY (US); Camille Abboud, Rochester, NY (US)

Correspondence Address: NIXON PEABODY LLP - PATENT GROUP, CLINTON SQUARE, P.O. BOX 31051, ROCHESTER, NY, 14603-1051, US

(21) Appl. No.: 10/576,428

(22) PCT Filed: Oct. 22, 2004
(86) PCT No.: PCT/US04/35064

§ 371 Date: Aug. 31, 2006

Related U.S. Application Data
(60) Provisional application No. 60/513,523, filed on Oct. 22, 2003.

(51) Int. Cl. A61K 39/395

(52) U.S. Cl. 424/144.1; 424/155.1

(57) ABSTRACT

Methods and products are described for inducing B cell apoptosis, using antibody-induced apoptosis. Specifically, polyclonal antiserum or one or more monoclonal antibodies, either alone or in combination, as well as fragments or variants thereof are employed in the methods and products of the present invention. These antibodies, or fragments or variants thereof, are capable of binding to B cell surface markers under conditions effective to collectively or individually induce apoptosis of the contacted B cell. Consequently, the methods and products of the present invention can be used therapeutically to treat, or as a preventative agent to protect against, a B cell-related condition or disorder.

This application claims the priority benefit of provisional U.S. patent application Ser. No. 60/513,523 filed Oct. 22, 2003, which is hereby incorporated by reference in its entirety.

The present invention was made, at least in part, with funding received from the National Institutes of Health under grant number K08-AI01641-05. The U.S. government may retain certain rights in this invention.

### FIELD OF THE INVENTION

The present invention relates generally to the preparation and use of anti-thymocyte antiserum to induce B cell apoptosis.

### BACKGROUND OF THE INVENTION

It has been reported recently that polyclonal rabbit anti-human thymocyte globulin (rATG) can be used with plasmapheresis to treat antibody mediated renal allograft rejection (Shah et al., “Treatment of CD4 Positive Acute Humoral Rejection with Plasmapheresis and Rabbit Polyclonal Antithymocyte Globulin,” Transplantation 77(9):1399-1405 (2004)). rATG binds to multiple epitopes on the T cell surface and induces both apoptosis and complement mediated lysis. Because rATG is made with human thymocytes, it is unclear what mechanisms would account for its efficacy in treating humoral allograft rejection. One possibility is that rATG interferes with T cell dependent activation of alloreactive B cells by removing CD4⁺ T cell help. A second possibility is that antibodies contained in rATG bind cell surface proteins shared by B and T cells, initiating complement mediated B cell lysis. Indeed, rATG is known to contain antibodies directed at surface molecules shared between thymocytes and B cells including: anti-MHC Class I and II, anti-CD95, anti-CD28, and anti-CD45 (Dalakas, “Mechanism of Action of Intravenous Immunoglobulin and Therapeutic Considerations in the Treatment of Autoimmune Neurologic Diseases,” Neurology 51(6 Suppl):S2-8 (1998); Bonnefoy-Berard and Revillard, “Mechanisms of Immunosuppression Induced by Antithymocyte Globulins and OKT3,” J. Heart Lung Transplant 15(5):435-442 (1996); Bonnefoy-Berard et al., “Antibodies Against Functional Leukocyte Surface Molecules in Polyclonal Antilymphocyte and Antithymocyte Globulins,” Transplantation 51(3):669-673 (1991); Genestier et al., “Induction of Fas (Apo-1, CD95)-Mediated Apoptosis of Activated Lymphocytes by Polyclonal Antithymocyte Globulins,” Blood 91(7):2360-2368 (1998)). Such cross-reactivity is also a feature of other polyclonal anti-lymphocyte preparations, notably Minnesota ALG which was made by immunizing horses with human B cell lines but had strong anti-T cell activity (Bourdage and Hamlin, “Comparative Polyclonal Antithymocyte Globulin and Antilymphocyte/Antilymphoblast Globulin Anti-CD Antigen Analysis by Flow Cytometry,” Transplantation 59(8):1194-1200 (1995)).

A third and more intriguing hypothesis is that rATG contains antibodies directed against unique B cell surface markers that interfere with B cell activation and induce apoptosis (Bonnefoy-Berard et al., “Apoptosis Induced by Polyclonal Antilymphocyte Globulins in Human B-cell Lines,” Blood 83(4):1051-1059 (1994)). rATG is made by immunizing rabbits with unfractionated lymphocyte preparations isolated by Ficoll density gradient centrifugation from human pediatric thymii. While this population of “thymocytes” is made up predominantly of T cells in varying stages of differentiation, 2-6% of thymocytes are B, plasma, and dendritic cells (Akashi et al., “B Lymphopoiesis in the Thymus,” J. Immunol. 164(10):5221-5226 (2000); Isaacson et al., “The Human Thymus Contains a Novel Population of B Lymphocytes,” Lancet 2(8574):1488-1491 (1987)).

It would be desirable, therefore, to identify a polyclonal antiserum or mixed monoclonal antibody preparation containing antibodies specifically directed at B cell surface proteins linked to pro-apoptotic pathways. The present invention is directed to achieving this objective and otherwise overcoming the above-identified deficiencies in the art.

### SUMMARY OF THE INVENTION

A first aspect of the present invention relates to a method of inducing B cell apoptosis that includes the step of contacting a B cell with a polyclonal anti-thymocyte serum or at least one of a plurality of monoclonal antibodies, or effective fragments or variants thereof, that bind to B cell surface markers under conditions effective to induce apoptosis of the contacted B cell.

A second aspect of the present invention relates to a method of inducing apoptosis in myeloma cells that includes the step of contacting a myeloma cell with a polyclonal anti-thymocyte serum or at least one of a plurality of monoclonal antibodies, or effective fragments or variants thereof, that bind to a myeloma cell surface marker under conditions effective to induce myeloma cell apoptosis.

A third aspect of the present invention relates to a method of treating multiple myeloma that includes the steps of providing either (i) a polyclonal anti-thymocyte serum or (ii) at least one of a plurality of monoclonal antibodies that bind to a myeloma cell surface marker, or effective fragments or variants thereof, and administering to a patient experiencing multiple myeloma an amount of (i) or (ii) that is effective to destroy myeloma cells, thereby treating the multiple myeloma condition.

A fourth aspect of the present invention relates to a method of treating a B cell or plasma cell-related autoimmune disorder that includes the steps of providing either (i) a polyclonal anti-thymocyte serum or (ii) at least one of a plurality of monoclonal antibodies, or effective fragments or variants thereof, that bind to a B cell or plasma cell surface marker; and administering to a patient experiencing a B cell or plasma cell-related autoimmune disorder an amount of (i) or (ii) that is effective to destroy B cells or plasma cells responsible for the autoimmune disorder, thereby treating the B cell or plasma cell-related autoimmune disorder.

A fifth aspect of the present invention relates to a method of treating a patient for a B cell malignancy that includes the steps of providing either (i) a polyclonal anti-thymocyte serum or (ii) at least one of a plurality of monoclonal antibodies, or effective fragments or variants thereof, that bind to a malignant B cell surface marker, and administering to a patient experiencing a B cell malignancy an amount of (i) or (ii) that is effective to destroy malignant B cells, thereby treating the patient for the B cell malignancy.

A sixth aspect of the present invention relates to a method of treating B cell or plasma cell-related alloantibody disorders in solid organ or bone marrow transplantation, said method including the steps of providing either (i) a polyclonal anti-thymocyte serum or (ii) at least one of a plurality of monoclonal antibodies, or effective fragments or variants thereof, that bind to a B cell or plasma cell surface marker on B cells or plasma cells that are implicated in an alloantibody disorder; and administering to a patient experiencing a B cell or plasma cell-related autoimmune disorder an amount of (i) or (ii) that is effective to destroy B cells or plasma cells responsible for the autoimmune disorder, thereby treating the B cell or plasma cell-related alloantibody disorder.

A seventh aspect of the present invention relates to a composition that includes two or more monoclonal antibodies or fragments or variants thereof that are effective in binding to a B cell or plasma cell surface marker, and either individually or collectively inducing apoptosis to the bound cell.

The present invention demonstrates that polyclonal anti-thymocyte antiserum induces apoptosis in naive and activated human B cells and plasma cells. Using competitive inhibition of monoclonal antibody binding, several B cell surface marker specificities of polyclonal anti-thymocyte were identified, many of which are known to induce B cell apoptosis. Ligating these surface proteins with monoclonal antibodies induces lesser degrees of B cell apoptosis than the polyclonal anti-thymocyte preparation, yet collective or pooled monoclonal antibody preparation should replicate the activity of the polyclonal anti-thymocyte antiserum. Finally, the experimental evidence demonstrates that apoptosis induced by the polyclonal anti-thymocyte antiserum involves three apoptotic pathways, including caspase activation, cathepsin B release from lysosomes, and mitochondrial membrane depolarization.

### BRIEF DESCRIPTION OF THE DRAWINGS

FIGS. 1 illustrates the induction of apoptosis as measured by four independent assays comparing rabbit anti-thymoglobulin (rATG), human intravenous immunoglobulin (IVIG), rituximab (anti-CD20), and alemtuzumab (anti-CD52). CD40L activated B cells were incubated with 100 μg/ml of rATG and assayed at 18 hours. Measurement of annexin V binding, subdiploid DNA fractionation, caspase 3 activation and loss of mitochondrial membrane polarization in CD40L stimulated B cells incubated for 18 hours with the indicated agents.

FIGS. 2A illustrates the induction of B cell apoptosis by rATG for naïve B cells, CD40-ligand stimulated B cells, and human bone-marrow resident CD138⁺ plasma cells. Dose-response curves of the efficacy of apoptosis induction by rATG, human intravenous immunoglobulin (IVIG), alemtuzumab (anti-CD52) and rituxumab (anti-CD20) are shown for CD40-ligand stimulated B cells (FIG. 2C) and human bone-marrow resident CD138⁺ plasma cells (FIG. 2B). Cells were incubated with varying concentrations of rATG (or IVIV, or alemtuzumab, or rituxumab) and apoptosis assayed after 18 hours by staining for annexin V followed by flow cytometry.

FIG. 3 illustrates the differential expression of B cell surface markers over different states of activation and differentiation. Naïve peripheral blood B cells (grey line) and CD40L activated peripheral blood B cells (black line) from normal volunteers were stained with monoclonal antibodies directed against the indicated proteins and analyzed by flow cytometry. Note the decreased expression of CD20 and CD52 on activated B cells.

FIG. 4 is a graph illustrating pre-plasmablast apoptosis induced by at least two mechanisms. Plasmablasts were derived by culture of CD19⁺ PBMC's with CD40L and IL-4. Cells were incubated with rATG (100 mcg/ml) for 18 hours in the presence of the indicated enzymatic inhibitors. The caspase inhibitor z-VAD-fmk, and the cathepsin B inhibitor E64d partially inhibited plasmablast apoptosis, indicating at least two mechanisms (caspase and cathepsin dependent) of plasmablast apoptosis. Calpain inhibitors, cathepsin D inhibitor, MAP/ERK 1,2 inlubitor, p38 MAPK inhibitor, MAPK inhibitor, serine protease inhibitor, and cysteine protease inhibitor did not significantly alter apoptosis.

FIGS. 5A illustrates the binding of monoclonal antibody targeted to the indicated marker (CD38 or HLA-ABC) in the presence of rATG. FIG. 5B shows the result of competitive inhibition of specific antibody binding by pre-incubation with rATG (100 mcg/ml) followed by staining with monoclonal antibodies directed against B cell and plasma cell surface markers. CD40L activated plasmablasts were used for all binding studies except CD138, for which the U-266 myeloma cell line was used.

FIG. 6 illustrates the results of a comparison of antibody induced apoptosis for monoclonal antibodies directed at B cell targets versus rATG.

FIG. 7A demonstrates immunohistochemistry of CD20⁺ B cells and CD138⁺ plasma cells in a normal human thymus. FIG. 7B shows flow cytometric analysis of intracellular κ and λ thymic populations, demonstrating B cells present in the pediatric thymus.

FIG. 8A is a graph illustrating the effect of rATG on myeloma cell lines. Myeloma cell lines were incubated with clinically relevant concentrations of rATG in complement free medium. Cells were assayed for apoptosis after 18 hours by flow cytometry and staining with Annexin V/TOPRO-3. rATG induced high levels of apoptosis in all myeloma cell lines, although two lines had less than 50% apoptosis at maximal concentrations. FIG. 8B is a graph illustrating the effect of rATG on bone marrow aspirates from patients with multiple myeloma, which were purified by Ficoll density gradient centrifugation and plasma/myeloma cells by CD138 affinity column positive selection. Cells were incubated with rATG 100 mcg/ml and apoptosis measure by Annexin V/TOPRO-3 staining. F=from frozen specimens, P=freshly isolated cells, (%)=percentage of marrow infiltrated with malignant cells.

FIG. 9 is a graph illustrating the induction of caspase-3 by rATG. CD138⁺ cells selected from myeloma cell lines were incubated in 100 ng/ml rATG or rabbit IgG (control), rituxumab, or campath® (alemtuzumab) in complement free medium. rATG induced caspase-3 at substantially higher levels than control, rituxumab, or alemtuzumab.

### DETAILED DESCRIPTION OF THE INVENTION

The present invention generally relates to methods and products for inducing B cell apoptosis, using antibody-induced apoptosis. Specifically, polyclonal antiserum or one or more monoclonal antibodies, either alone or in combination, as well as fragments and variants thereof, are employed in the methods and products of the present invention. These antibodies or fragments or variants thereof are capable of binding to B cell surface markers under conditions effective to induce apoptosis of the contacted B cell. Consequently, the methods and products of the present invention can be used therapeutically to treat, or as a preventative agent to protect against, a B cell-related condition or disorder.

As used herein, B cell generally refers in the broadest sense to all cells derived from the B cell lineage. More particularly, these can include, without limitation, one or more of immature B cells, naïve B cells, activated B cells, memory B cells, blastic B cells, plasma cells, and mixed populations of any combination of those cells. Specific types of B cells can be further differentiated based upon cell surface markers that they possess. Exemplary B cells that can be treated in accordance with the present invention include, without limitation CD16, CD19, CD20, CD27, CD30, CD32, CD38, CD40, CD45, CD80, CD86, CD95, CD138, HLA-A, HLA-B, HLA-C, HLA-DR, HLA-DQ, HLA-DP, MHC Class I, MHC Class II, sIgG, sIgM, sIgD, sIgE, and sIgA, hyaluronic acid receptor, alpha interferon receptor, Ig Kappa-or lambda-light chain, Ig heavy chain, and TNF proteins. Preferred B cells that can be treated in accordance with the present invention include, without limitation, CD19⁺ peripheral blood B cells of memory or naïve phenotype, CD40L activated B cell plasmablasts of memory or naïve phenotype, and/or normal human plasma cell.

As used herein, anti-B cell antibodies or fragments or variants thereof include antibodies and fragment or variants thereof that have been raised against either a thymic tissue sample (including one or more types of B cells and plasma cells), selected B cell populations (optionally excluding normal plasma cells), or isolated and purified cell surface receptors. Consequently, the anti-B cell antibodies, or fragments or variants thereof, can recognize and bind to the antigen against which they were raised. The anti-B cell antibodies can be either monoclonal or polyclonal antibodies, or a mixed population of monoclonal antibodies, as well as fragments or variants thereof that retain their ability to bind to a B cell surface marker and induce apoptosis of the B cell. The polyclonal and monoclonal antibodies can be raised from any species, including genetically modified animals, or derived from in vitro antibody production techniques, as described hereinafter.

Monoclonal antibody production may be effected by techniques which are well-known in the art. Basically, the process involves first obtaining immune cells (lymphocytes) from the spleen of a mammal (e.g., mouse, rat, rabbit, pig, non-human primate) which has been previously immunized with the antigen of interest (thymic tissue sample, selected B cell populations, or isolated and purified cell surface receptors) either in vivo or in vitro. The antibody-secreting lymphocytes are then fused with myeloma cells or transformed plasma cells, which are capable of replicating indefinitely in cell culture, thereby producing an immortal, immunoglobulin-secreting cell line. The resulting fused cells, or hybridomas, are cultured, and the resulting colonies screened for the production of the desired monoclonal antibodies. Colonies producing such antibodies are cloned and grown either in vivo or in vitro to produce large quantities of antibody. A description of the theoretical basis and practical methodology of fusing such cells is set forth in Kohler and Milstein, Nature 256:495 (1975), which is hereby incorporated by reference in its entirety.

Mammalian lymphocytes are immunized by in vivo immunization of the animal (e.g., a mouse, rat, rabbit, pig, or primate) with thymic tissue sample, selected B cell populations, or isolated and purified cell surface receptors (as described above). Such immunizations are repeated as necessary at intervals of up to several weeks to obtain a sufficient titer of antibodies. Following the last antigen boost, the animals are sacrificed and spleen cells removed.

Fusion with mammalian myeloma cells or other fusion partners capable of replicating indefinitely in cell culture is effected by standard and well-known techniques, for example, by using polyethylene glycol (“PEG”) or other fusing agents (see Milstein and Kohler, Eur. J. Immunol. 6:511 (1976), which is hereby incorporated by reference in its entirety). This immortal cell line, which is preferably murine, but may also be derived from cells of other mammalian species, including but not limited to rats, pigs, non-human primates, and humans, is selected to be deficient in enzymes necessary for the utilization of certain nutrients, to be capable of rapid growth, and to have good fusion capability. Many such cell lines are known to those skilled in the art, and others are regularly described. Human hybridomas can be prepared using the EBV-hybridoma technique monoclonal antibodies (Cole et al., in Monoclonal Antibodies and Cancer Therapy, Alan R. Liss, Inc., pp. 77-96 (1985), which is hereby incorporated by reference in its entirety). Human antibodies may be used and can be obtained by using human hybridomas (Cote et al., Proc. Natl. Acad. Sci. USA 80:2026-2030 (1983), which is hereby incorporated by reference in its entirety) or by transforming human B cells with EBV virus in vitro (Cole et al., in Monoclonal Antibodies and Cancer Therapy, Alan R. Liss, Inc., pp. 77-96 (1985), which is hereby incorporated by reference in its entirety). In addition, monoclonal antibodies can be produced in germ-free animals (see PCT/US90/02545, which is hereby incorporated by reference in its entirety).

Procedures for raising polyclonal antibodies are also well known. Typically, such antibodies can be raised by administering the antigen (as described above) subcutaneously to rabbits, mice, rats, pigs, or non-human primates which have first been bled to obtain pre-immune serum. The antigens can be injected as tolerated. Each injected material can contain adjuvants and the antigen (preferably in substantially pure or isolated form, depending on procedures employed therefor). Suitable adjuvants include, without limitation, Freund's complete or incomplete, mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as bacille Calmette-Guerin, or CpG DNA. The subject mammals are then bled one to two weeks after the first injection and periodically boosted with the same antigen (e.g., three times every six weeks). A sample of serum is then collected one to two weeks after each boost Polyclonal antibodies can be recovered from the serum by affinity chromatography using the corresponding antigen to capture the antibody. This and other procedures for raising polyclonal antibodies are disclosed in Harlow & Lane, editors, Antibodies: A Laboratory Manual (1988), which is hereby incorporated by reference in its entirety.

In addition, techniques developed for the production of chimeric antibodies (Morrison et al., Proc. Natl. Acad. Sci. USA 81:6851-6855 (1984); Neuberger et al., Nature 312:604-608 (1984); Takeda et al., Nature 314:452-454 (1985), each of which is hereby incorporated by reference in its entirety) by splicing the genes from a mouse antibody molecule of appropriate antigen specificity together with genes from a human antibody molecule of appropriate biological activity can be used. For example, the genes from a mouse antibody molecule specific for thymic tissue sample, selected B cell populations, or isolated and purified cell surface receptors can be spliced together with genes from a human antibody molecule of appropriate biological activity. A chimeric antibody is a molecule in which different portions are derived from different animal species, such as those having a variable region derived from a murine mAb and a human immunoglobulin constant region (e.g., U.S. Pat. No. 4,816,567 to Cabilly et al.; U.S. Pat. No. 4,816,397 to Boss et al., each of which is hereby incorporated by reference in its entirety).

In addition, techniques have been developed for the production of humanized antibodies (e.g., U.S. Pat. No. 5,585,089 to Queen; U.S. Pat. No. 5,225,539 to Winter, each of which is hereby incorporated by reference in its entirety). An immunoglobulin light or heavy chain variable region consists of a “framework” region interrupted by three hypervariable regions, referred to as complementarity determining regions (CDRs). The extent of the framework region and CDRs have been precisely defined (see Kabat et al., “Sequences of Proteins of Immunological Interest,” U.S. Department of Health and Human Services (1983), which is hereby incorporated by reference in its entirety). Briefly, humanized antibodies are antibody molecules from non-human species having one or more CDRs from the non-human species and a framework region from a human immunoglobulin molecule.

Alternatively, techniques described for the production of single chain antibodies (e.g., U.S. Pat. No. 4,946,778 to Ladner et al.; Bird, Science 242:423-426 (1988); Huston et al., Proc. Natl. Acad. Sci. USA 85:5879-5883 (1988); Ward et al., Nature 334:544-546 (1989), each of which is hereby incorporated by reference in its entirety) can be adapted to produce single chain antibodies against thymic tissue sample, selected B cell populations, or isolated and purified cell surface receptors. Single chain antibodies are formed by linking the heavy and light chain fragments of the Fv region via an amino acid bridge, resulting in a single chain polypeptide.

In addition to utilizing whole antibodies, the present invention also encompasses use of binding portions of such antibodies. Such binding portions include Fab fragments, F(ab′)₂ fragments, and Fv fragments. These antibody fragments can be made by conventional procedures, such as proteolytic fragmentation procedures, as described in Goding, Monoclonal Antibodies: Principles and Practice, pp. 98-118, New York:Academic Press (1983), which is hereby incorporated by reference in its entirety. Alternatively, the Fab fragments can be generated by treating the antibody molecule with papain and a reducing agent. Alternatively, Fab expression libraries maybe constructed (Huse et al., Science 246:1275-1281 (1989), which is hereby incorporated by reference in its entirety) to allow rapid and easy identification of monoclonal Fab fragments with the desired specificity.

The above-identified antibodies may be isolated by standard techniques known in the art such as immunoaffinity chromatography, centrifugation, precipitation, etc. The antibodies (or fragments or variants thereof) are preferably prepared in a substantially purified form (i.e., at least about 85 percent pure, more preferably 90 percent pure, even more preferably at least about 95 to 99 percent pure).

In an embodiment of the invention, molecules comprising the binding portion of antibodies which specifically bind to thymic tissue sample, selected B cell populations, or isolated and purified cell surface receptors may be used in the methods of the invention.

A preferred polyclonal anti-thymic cell antiserum is rabbit-thymoglobulin (rATG) (SangStat Medical Corp., Fremont, Calif.).

Preferred monoclonal antibody preparations include at least two or more, at least three or more, at least four or more, at least five or more, at least six or more, at least seven or more, at least eight or more, at least nine or more, or at least ten or more apoptosis-inducing anti-B cell monoclonal antibodies. Each of the monoclonal antibodies that form the preferred preparations of the present invention can be capable of binding to one of the above-identified cell surface markers and inducing apoptosis of B cells to which they bind.

The anti-B cell antibodies prepared by the methods of the present invention can also be utilized as the biologically active components in pharmaceutical compositions.

The pharmaceutical composition can also include, but are not limited to, suitable adjuvants, carriers, excipients, or stabilizers, and is preferably though not necessarily in liquid form such as solutions, suspensions, or emulsions. Typically, the composition will contain from about 0.01 to 99 percent, preferably from about 20 to 75 percent of the antibodies or fragments or variants thereof, together with the adjuvants, carriers, excipients, stabilizers, etc.

The pharmaceutical forms suitable for injectable use include sterile aqueous solutions or dispersions and sterile powders for the extemporaneous preparation of sterile injectable solutions or dispersions. In all cases, the form should be sterile and should be fluid to the extent that easy syringability exists. It should be stable under the conditions of manufacture and storage and should be preserved against the contaminating action of microorganisms, such as bacteria and fungi. Suitable adjuvants, carriers and/or excipients, include, but are not limited to sterile liquids, such as water and oils, with or without the addition of a surfactant and other pharmaceutically and physiologically acceptable carrier, including adjuvants, excipients or stabilizers. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions.

In the pharmaceutical compositions of the present invention, the antibodies or fragments or variants thereof are preferably present at a concentration of about 0.1 to about 100 mg/ml, more preferably about 1 to about 10 mg/ml, and even more preferably about 1 to about 5 mg/ml, and administered in a sufficient dose to obtain serum concentrations of about 50 to about 400 mcg/ml as measured by ELISA serum within 24 hours of administration.

The anti-B cell antibodies (or fragments or variants thereof) are to be administered in an amount effective to achieve their intended purpose. While individual needs vary, determination of optimal ranges of effective amounts of each component is within the skill of the art. The quantity administered will vary depending on the patient and the mode of administration and can be any effective amount. Typical dosages include about 0.1 to about 100 mg/kg-body wt. The preferred dosages include about 1 to about 3 mg/kg-body wt on day 1-5 of a 21-day course of therapy. However, because patients respond differently to therapies, administration of the anti-B cell antibodies or fragments or variant thereof can be adjusted following monitoring of serum levels for purposes of optimizing therapeutic effects. Treatment regimen for the administration of the antibodies or fragments or variants of the present invention can also be determined readily by those with ordinary skill in art.

Depending upon the treatment being effected, the antibodies or compositions of the present invention can be administered orally, topically, transdermally, parenterally, subcutaneously, intravenously, intramuscularly, intraperitoneally, by intranasal instillation, by intracavitary or intravesical instillation, intraocularly, intraarterially, intralesionally, or by application to mucous membranes, such as, that of the nose, throat, and bronchial tubes. Of these routes, intravenous administration is preferred. Administration can be periodically repeated to achieve optimal apoptotic effect upon the targeted B cells.

The anti-B cell antibodies, as well as fragment or variants thereof, are intended to be used for either in vitro or in vivo induction of B cell apoptosis for the treatment or prevention of B cell-related disorders. B cell-related disorders that can be treated and/or prevented can generally be defined as B cell or plasma cell-related autoimmune disorders as well as alloantibody disorders in solid organ or bone marrow transplantation. Exemplary disorders of these types include, without limitation, systemic lupus erythematosus, Rheumatoid arthritis, diabetes, Sjogren's syndrome, Hashimoto's disease, Wegner's granulomatosis, polyarteritis nodosum, anti-cardiolipin antibody syndrome, autoimmune hepatitis, B cells cancers of the immune system (such as non-Hodin's lymphoma and multiple myeloma). With respect to the treatment of B cell cancers, it is possible to treat a patient for B cell malignancies (by inducing apoptosis of malignant B cells). With respect to the treatment of B cell alloantibody disorders, it is possible to treat a patient to eliminate antibodies from the recipient that are directed at, and toxic to, the transplanted organ or tissue by inducing apoptosis of the B cells and/or plasma cells producing those antibodies using the present invention in combination with other therapies including, but not limited to, plasmapheresis, apheresis, intravenous immunoglobulin, and concurrent immunosuppression.

### EXAMPLES

The following examples are intended to illustrate, but by no means are intended to limit, the scope of the present invention as set forth in the appended claims.

Materials and Methods

Human Subjects Protection: This study was approved by the Research Subjects Review Board at the University of Rochester Medical Center. Informed consent was obtained from all participants. Research data were coded such that subjects could not be identified, directly or through linked identifiers, in compliance with the Department of Health and Human Services Regulations for the Protection of Human Subjects (45 CFR 46.101(b)(4)).

Peripheral Blood B Cell and Bone Marrow Plasma Cell Isolation: PBMCs and CD19+ B cells were isolated from peripheral blood of normal human volunteers and cultured as previously described (Shah et al., “Treatment of CD4 Positive Acute Humoral Rejection With Plasmapheresis and Rabbit Polyclonal Antithymocyte Globulin,” Transplantation 77(9):1399-405 (2004); Zand et al. “A Renewable Source of Donor Cells for Repetitive Monitoring of T and B Cell Alloreactivity,” Am. J. Transpl. (electronically published Oct. 13, 2004, Blackwell Synergy Internet site), each of which is hereby incorporated by reference in its entirety). CD19⁺ cells were negatively selected from PBMCs that were incubated with magnetic beads coupled to anti-CD3, CD11b, CD16, CD36 and CD56 (Miltenyi, Auburn Calif.). Purified CD19⁺ B cells were activated with CD40L and recombinant human IL-4 to make B cell blasts (see below). Naïve CD19⁺ CD27⁺ B cells were isolated by negative selection with anti-CD27 coupled magnetic beads and used immediately for experiments. Bone marrow resident plasma cells were isolated from bone marrow aspirates of normal human volunteers. Aspirates were diluted 1:1 in PBS and cells isolated by Ficoll density gradient centrifugation. CD138⁺ plasma cells were isolated by positive selection using anti-CD138 coupled magnetic beads and a magnetic affinity column. Cell purity for all isolations was >98%, and verified by FACS staining.

Culture of CD40L Stimulated B Cell Blasts: Unfractionated CD19⁺ B cells were used to make CD40L⁺ IL-4 activated B cell blasts as previously described (Zand et al. “A Renewable Source of Donor Cells for Repetitive Monitoring of T and B Cell Alloreactivity,” Am. J. Transpl. (electronically published Oct. 13, 2004, Blackwell Synergy Internet site), which is hereby incorporated by reference in its entirety). Briefly, CD19⁺ B cells were grown on a feeder layer of NIH-3T3 cells transfected with human CD40L irradiated with 96 cGy in 6 well plates a density of 5×10⁵ cells/well in Iscove's MDM (Gibco/BRL) with 10% heat inactivated human AB serum (Sigma), 50 μg/ml human transferrin (Boehringer Mannheim), 5 μg/ml human insulin (Sigma), 15 μg/ml gentamicin (Gibco/BRL), 8 ng/ml recombinant human IL-4 (Pharmingen) and 5.5×10⁻⁷ M cyclosporine A (Sigma) (Schultze et al., “CD40-activated Human B Cells: An Alternative Source of Highly Efficient Antigen Presenting Cells to Generate Autologous Antigen-Specific T Cells for Adoptive Immunotherapy,” J. Clin. Invest. 100(11):2757-2765 (1997), which is hereby incorporated by reference in its entirety). All lots of human sera used in cell culture or experiments was tested for the absence of human complement activity prior to use. After 1 week, cells were cultured in cyclosporine free medium at a density of 2.5×10⁶ cells/well. Prior to experiments, dead sBc cells and residual fibroblasts were removed by Ficoll density gradient centrifugation.

B Cell culture: Human B cells were cultured from peripheral blood as previously described. Mononuclear cells were isolated by Ficoll density gradient centrifugation and plated in 6 well plates at a density of 4×10⁶ cells/well in Isocove's MDM (Giboco/BRL) with 10% heat inactivated human AB serum (Sigma), 50 μg/ml human transferrin (Boehringer Mannheim), 5 μg/ml human insulin (Sigma), 15 μg/ml gentamicin (Gibco/BRL), 8 ng/ml recombinant human IL-4 (Pharmingen) and cyclosporine A (5.5×10⁻⁷ M). After 1 week in culture, cells were passaged at a density of 2.5×10⁶ cells per well without cyclosporine A. The B cells were grown on a feeder layer of NIH-3T3 cells transfected with human CD40L which have been irradiated (96 cGy) and plated at a density of 10⁵ cells per well. B cells were frozen in a medium of 90% AB serum and 10% DMSO. Prior to apoptosis assays, B cells were re-isolated by Ficoll density gradient centrifugation to remove dead cells and residual fibroblasts.

Plasma cell isolation and culture: Human plasma cells were isolated from bone marrow aspirates of normal human donors by Ficoll-Plaque density gradient centrifugation followed by positive selection using anti-CD138 immunomagnetic beads (Miltenyi). Cell isolates were 99.5% pure as assessed by flow cytometry. Plasma cells were cultured in Isocove's MDM (Gibco/BRL) with 10% heat inactivated fetal bovine serum (Sigma), 50 μg/ml human transferrin, 5 μg/ml human insulin, 15 μg/ml gentamicin, 8 ng/ml recombinant human IL-4. All lots of fetal bovine sera used in cell culture or experiments was tested for the absence of human complement activity prior to use.

Isolation of Human Lymphocytes: Lymphocytes were isolated by Ficoll density gradient centrifugation from either the peripheral blood of healthy volunteers, or from cadaveric organ donor spleen or lymph node tissue. CD4⁺ and CD8⁺ responder cells were purified by negative selection for non-T cells using antibody-coupled (anti-human CD4 or CD8, CD11b, CD16, CD19, CD36 and CD56) magnetic bead columns (Miltenyi). Isolated T cell subsets were verified to be 99% pure by flow cytometric analysis. Cells were then activated with plate bound anti-CD3 and anti-CD28 in the presence of 20 U/ml recombinant human IL-2 (Pharmingen).

Antibody Reagents and Flow Cytometry: Flow cytometric analysis was performed with a FacsCaliber dual laser cytometer (Becton-Dickinson) using CellQuest (Becton-Dickinson) acquisition and Cytomation (Summit) analysis software. The following antibodies were used for staining (murine monoclonal from BD Pharmingen, unless otherwise noted): fluorescein conjugated goat F(ab′)₂ (anti-heavy chain) anti-human IgG Fc (Jackson ImmunoResearch), unconjugated goat F(ab′)₂ anti-human heavy and light chain IgG (Jackson ImmunoResearch), anti-CD3 PE (clone HIT3a), flourescein conjugated and unconjugated human IgG Fc fragments (Jackson ImmunoResearch), goat anti-human anti-IgD (Southern Biotech), biotin anti-CD4 (clone RPA-T4), biotin anti-CD8⁺ (RPA-T8), anti-human CD16 (3G8), PE anti-CD19 (HIB19), PE and CyChrome anti-CD20 (2H7), FITC anti-CD27 (M-T271), unconjugated and PE anti-CD32 (FL18.26), CyChrome anti-CD38 (HIT2), PE anti-CD40 (5C3), PE anti-IgD (I45.2), PE anti-CD40, PE anti-HLA-A,B,C (G46-2.6), CyChrome (TU36) and unconjugated (G46-6) anti-HLA-DR, PE anti-CD16 (3G8), PE anti-CD52 (Serotec; YTH34.5), PE anti-CD80 (L307.4), biotin anti-CD86 (IT2.2), PE anti-CD95 (DX2), biotin anti-CD64 (10.1), PE anti-human kappa chain (TB28-2), and FITC anti human lambda chain (I-155-2). PE, FITC or CyChrome conjugated murine IgG₁êand IgG₂ê, or rat IgG_{2b} were used as isotype controls. PE and CyChrome conjugated streptavidin (BD Pharmingen) were used as second-step reagents for biotinylated antibodies.

Rabbit IgG (Sigma, Mo.), anti-thymocyte globulin rabbit and anti-human thymoglobulin (Sangstat, Fremont Calif.), rituximab (IDEC Pharmaceuticals, Canbridge, Mass.), alumtizumab (Berlex, Calif.) were reagents used in the induction of apoptosis. rATG was generously provided by Sangstat/Genzyme, or obtained independently by the investigators. Critical experiments were verified across four different lots of rATG. Caspase substrates z-VAD-fmk, z-FA-fmk, were from Cell Technology, Inc. (Mountain View, Calif.). Mitochondrion-selective probe tetramethylrhodamine methyl ester (TMRM) was purchased from Molecular Probes, Inc. (Eugene, Oreg.). Annexin V and TOPRO-3 were from BD Biosciences Pharmigen and Molecular Probes, Inc., respectively.

Measurement of apoptosis: Induction and measurement of apoptosis was performed on naïve peripheral blood B cells (CD19⁺ CD27), CD40L stimulated B-cells (CD19⁺, CD27⁺, CD38⁺, HLA-ABCₕᵢ, HLA-DRₗₒ), CD3⁺ T-cells three days after activation by anti-CD3 and anti-CD28, and normal human plasma cells (CD138⁺). For each experiment, 10⁶ cells/well were cultured in 96-well flat-bottom plates in their respective medium. To test their capacity for induction of apoptosis, the following agents were added to the medium: rATG (0.0001-1.0 mg/ml), rituximab (0.001-10 mg/ml), alemtizumab (0.001-1.0 mg/ml), or with rabbit IgG (0.0001-1.0 mg/ml) as a negative control. Cells were then incubated for a specified time period at 37° C. For some experiments, inhibitors of apoptosis pathways were added 1 hour prior to addition of the rATG or antibodies at the following concentrations:

Induction of apoptosis was assessed by the following four methods.

(1) Caspase induction was measured by adding fluorescently tagged substrates for caspase 3 (z-DEVD-fmk), caspase 8 (z-IETD-fmk), or caspase 9 (z-LEHD-fmk) to cell culture medium at a final concentration of 1 μg/ml one hour prior to flow cytometry Caspase induction was assessed by FL2 channel shift. Experiments included controls with the non-labeled pan-caspase inhibitor z-VAD-FMK (100 μg/ml).

(2) Loss of mitochondial membrane potential was measured by quantifying the fluorescence intensity of the mitochondrion-selective probe tetramethylrhodamine methyl ester (TMRM; Molecular Probes, Eugene Oreg.) which is taken up by depolarized mitochondria TMRM was added to the culture medium one half hour to prior to analysis, and cells were washed with PBS with 1% BSA (no sodium azide) prior to analysis.

(3) Loss of plasma membrane polarity was assessed by flow cytometric analysis of annexin-V. Cells were washed twice in PBS+BSA buffer and labeled with FITC conjugated annexin-V in the presence of the DNA binding dye TOPRO-3 (1 ng/ml) and immediately analyzed by flow cytometry.

(4) Subdiploid DNA fragmentation was assessed by fixing cells in 70% methanol, resuspending cells in PBS+BSA, incubating with DNAase-free RNAase (10 U/ml, 30 minutes at 37° C.), and staining with TOPRO-3. DNA content was assessed by flow cytometry.

Assay of B cell specific antibodies in rATG: To assess if rATG contained specific antibodies directed against known B cell specific surface markers, competitive inhibition of binding by specific monoclonal antibodies was attempted via pre-incubating cells with rATG. After Ficoll density gradient centrifugation to remove dead cells and residual fibroblasts, CD40 ligand stimulated B-cells (sBc) or primary human plasma cells were washed twice and resuspended in staining buffer (PBS+1% BSA+0.01% NaAzide). 10⁶ cells/well were pre-incubated with either rabbit IgG or rATG (100 μg/ml) on ice for one hour. Cells were then washed with staining buffer and incubated with 10 μl human AB(−) serum for 15 minutes on ice. After washing, cells were probed with specific fluorochrome conjugated antibodies (IgG1 isotype control, αHLA-ABC, αHLA-DR, αCD-19, αCD-20, αCD-27, αCD-38, αCD-52, αCD-80, and αCD-95) on ice for 40 minutes. Cells were then washed twice and the pellets were resuspended in 500 μl of staining buffer and analyzed by flow cytometry as above.

Preparation of F(ab)₂ fragments of rATG: F(ab)₂ fragments of rATG and unimmunized rabbit IgG were prepared by pepsin digestion using the Immunopure F(ab)₂ kit (Pierce Chemical, Rockford, Ill.). Lyophilized rATG with vehicle was resuspended in sterile distilled water (20 mg/ml). For F(ab)₂ fragment preparation, resuspended rATG was extensively dialyzed against a 20 mM sodium acetate buffer (pH 4.5) and 0.5 ml was then added to an equal volume of digestion buffer (pH 4.5) and a slurry of immobilized pepsin and incubated at 37° C. for 5 hours. The slurry was centrifuged and the supernatant passed over a protein A column to bind undigested Ig and Fc fragments. F(ab)₂ fragments were eluted in the unbound column fraction as assessed by absorbance at 280 nm and extensively dialyzed against PBS (pH 7.0). Digestion was confirmed by polyacrylamide gel electrophoresis. The final F(ab)₂ reagent was used at concentrations equimolar to that of rATG concentration of 5 mg/ml. Fragments of control unimmunized rabbit Ig were prepared in a similar fashion.

Immunohistochemical staining of thymic tissue: Paraffin embedded sections of human pediatric thymuses were selected from extant tissue blocks 72 normal thymii removed from patients less than ten years old during 2001. A random sample of 10 blocks was selected and new cut sections were cut. Immunoperoxidase staining was performed by previously published methods (Chilosi et al., “CD138/syndecan-1: A Useful Immunohistochemical Marker of Normal and Neoplastic Plasma Cells on Routine Trephine Bone Marrow Biopsies,” Mod. Pathol. 12(12):1101-1106 (1999); Komrokji et al., “Burkitt's Leukemia With Precursor B-cell Immunophenotype and Atypical Morphology (Atypical Burkitt's Leukemia/Lymphoma): Case Report and Review of Literature,” Leuk. Res. 27(6):561-566 (2003), each of which is hereby incorporated by reference in its entirety) using a streptavidin-biotin detection system, horseradish peroxidase, and 7-aminoethylcarbizole (7-AEC) as the substrate. The primary antibodies were: CD3 (1:100 primary dilution, DAKO, Carpinteria, Calif.), CD20 (1:800 dilution, clone L26, DAKO) and CD138 (1:100, syndecan-1, clone B-B4, Serotec, Kidlingston, UK) (Chilosi et al., “CD138/syndecan-1: A Useful Immunohistochemical Marker of Normal and Neoplastic Plasma Cells on Routine Trephine Bone Marrow Biopsies,” Mod. Pathol. 12(12):1101-1106 (1999), which is hereby incorporated by reference in its entirety).

### Example 1

### Induction of B cell apoptosis by rATG

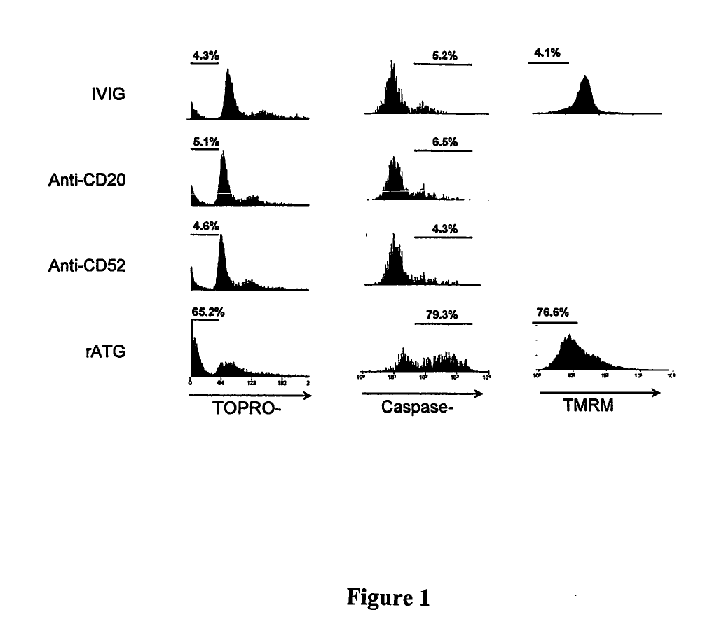

The ability of rATG to induce apoptosis in B cells was determined using four different assays (FIG. 1): loss of plasma membrane polarization by annexin V binding to the outer leaflet, subdiploid DNA content, caspase 3 induction, and loss of mitochondrial membrane potential measured by uptake of the dye TMRM. Incubation of rATG with CD40L activated B cells at increasing concentrations demonstrated a progression from live (Annexin_{neg}, TOPRO_{neg}), to apoptotic (Annexinₚₒₛ, TOPRO_{neg}), and finally late apopototic (Annexinₚₒₛ, TOPROₚₒₛ) phases. Several clinical protocols have been described for treatment or prevention of antibody mediated allograft rejection using IVIG, rATG, rituxumab, and alemtizumab. This panel of assays was therefore used to compare the induction of apoptosis for each of these agents at clinically relevant concentrations. rATG was the only agent to induce significant apoptotic change in all four assays.

### Example 2

### Dose-Response Curves for Human Naïve and Activated B Cells, and Plasma Cells

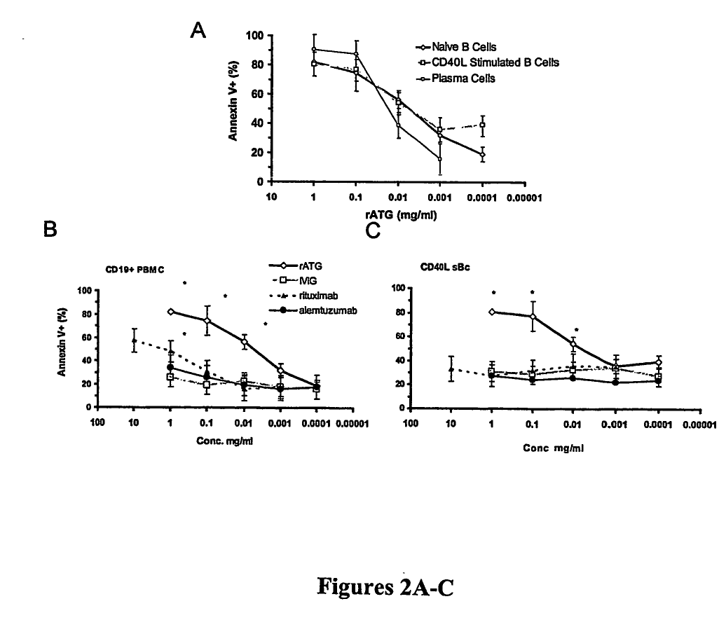

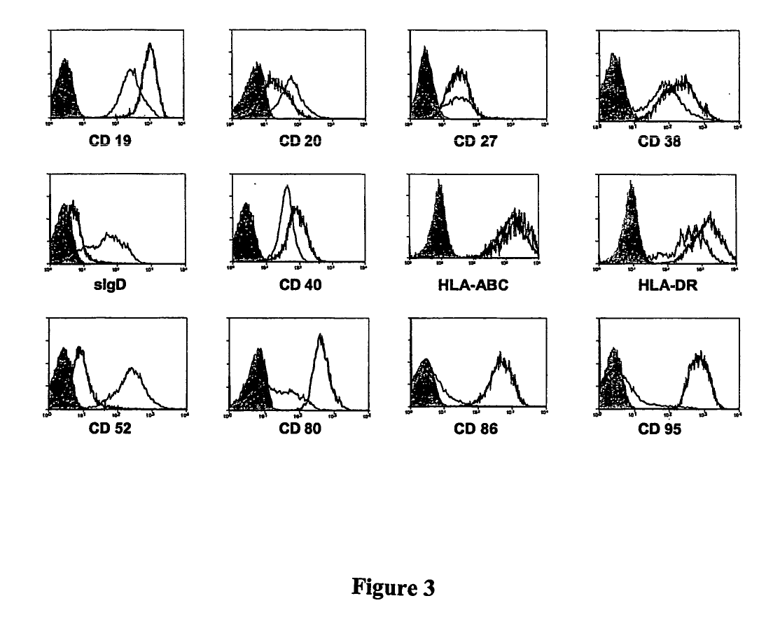

Because B cells at varying stages of activation both express different surface markers (FIG. 2) and have varying sensitivity to antibody-mediated apoptosis, the ability of rATG to induce apoptosis in human naive B cells (CD20_{high} CD27⁻), activated B cells (CD20ₗₒ, CD27ₕᵢ) and normal bone marrow resident plasma cells (CD20⁻, CD138⁺) was tested. Cells were tested at clinically relevant range of rATG concentrations (1-1,000 μg/ml). All three cell types underwent dose-dependent induction of apoptosis with rATG (FIG. 2A). Given the differences in target antigen and Fc receptor expression between B cells of the naïve, activated and plasma cell phenotypes (See FIG. 3), an assessment was made as to the sensitivity of each of these subtypes to induction of apoptosis by other agents reported to have efficacy in treating antibody mediated allograft rejection or inducing B cell apoptosis. The sensitivity of each type of B cell to induction of apoptosis with human immunoglobulin (IVIG), rATG, rituxumab, and alemtizumab (FIGS. 2B-C) were measured. While naïve B cells were sensitive to rATG, rituxumab, and to a lesser extent alemtizumab, activated B cells and plasma cells were only sensitive to rATG.

### Example 3

### rATG F(ab)₂ Fragment Activity Against B Cells is Augmented by FcR Ligation

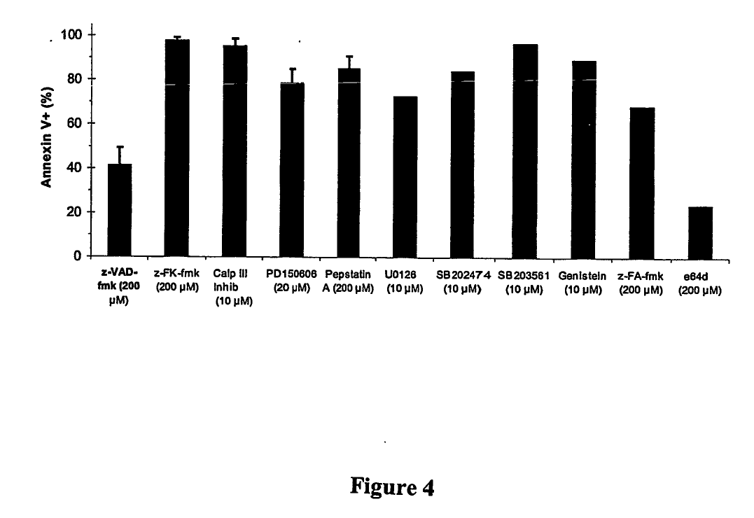

Binding of antigen-antibody complexes to B cell Fcγ receptors is known, under some circumstances, to induce B cell apoptosis. For example, FcRγ ligation augments monoclonal anti-CD95 mediated apoptosis (Xu et al. “Fc Gamma Rs Modulate Cytotoxicity of anti-Fas Antibodies: Implications for Agonistic Antibody-Based Therapeutics,” J. Immunol. 171(2):562-568 (2003), which is hereby incorporated by reference in its entirety) and causes accelerated apoptosis of B cells (Ashman et al., “Fc Receptor Off-Signal in the B Cell Involves Apoptosis,” J. Immunol. 157(1):5-11 (1996), each of which is hereby incorporated by reference in its entirety). It was therefore examined whether FcR binding augmented the degree of ATG induced apoptosis. Incubation of CD40L activated B cells with rATG F(ab)₂ fragments resulted in lower levels of apoptosis compared to the intact molecule (FIG. 4). While Fc ligation itself had little effect, co-incubation of rATG F(ab)₂ with anti-CD32, anti-CD64 or human Fc fragments essentially restored the full apoptotic activity of the F(ab)₂ fragments.

### Example 4

### Identifying the Targets of rATG on B Cells

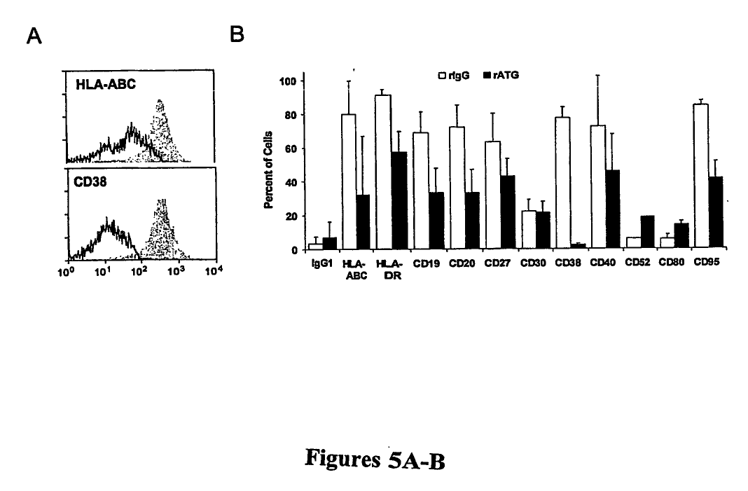

Several surface proteins expressed on B cells are known to induce apoptosis when cross-linked, including CD5, CD27, CD30, CD38, CD95, and HLA-DR. To determine if rATG contained antibodies directed at proteins linked to known B cell apoptotic pathways, competitive binding studies were performed. CD40L activated B cells were incubated in either rATG or control unimmunized rabbit Ig, followed by labeling with monoclonal antibodies specific for known pro-apoptotic B cell surface proteins, as well as several B cell specific markers (FIG. 5B). While a negative result in such assays does not exclude the possibility that rATG contains antibodies directed at an alternate epitope, a positive result strongly suggests the presence of antibodies directed against epitopes recognized by both monoclonal and polyclonal antibody preparations. Significant inhibition was observed for both HLA-A/B/C and HLA-DR, the B cell specific surface proteins CD19, CD20, CD80, CD40, as well as CD30, CD38, and CD95. Antibodies to CD38 and HLA-A1B1C are able to bind these markers even after exposure to rATG (FIG. 5A).

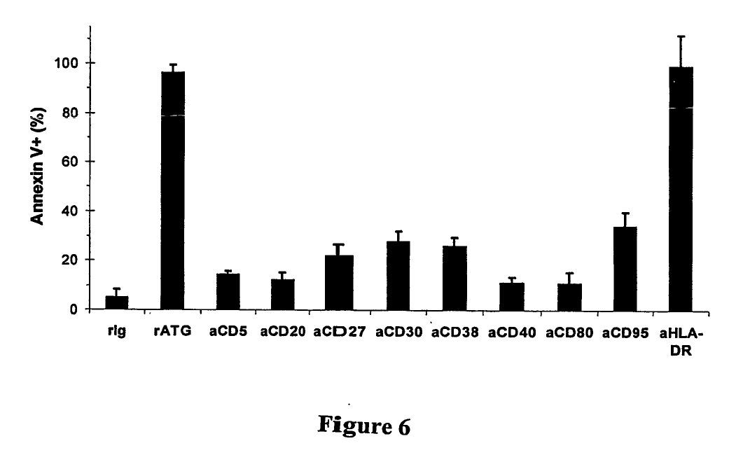

Next, an examination was made to assess whether ligation and crosslinking of these individual surface proteins with monoclonal antibodies could induce a degree of apoptosis similar to that of rATG (FIG. 6). CD40L activated B cells were cultured in 96 well plates coated with 100 μg/ml of either rATG, unimmunized rabbit IgG, or monoclonal antibodies against surface proteins known to be associated with B cell apoptosis pathways that had also been identified as likely constituents of rATG by the above competitive binding assays. Compared with controls, antibodies directed against CD27, CD30, CD38, CD95 and HLA-DR induced significant levels of apoptosis. Of note, anti-HLA-DR induced the highest levels of apoptosis, approaching those of rATG itself.

### Example 5

### Identifying the Apoptotic Pathways Associated with rATG in B Cells

The competitive binding experiments suggested significant rATG activity against CD20, CD30, CD38, and HLADR. Each of these surface proteins have been linked to specific apoptotic pathways: CD95 and CD38 induce caspase-dependent apoptosis (Blancheteau et al., “HLA Class II Signals Sensitize B Lymphocytes to Apoptosis Via Fas/CD95 by Increasing FADD Recruitment to Activated Fas and Activation of Caspases,” Hum. Immunol. 63(5):375-383 (2002); Leveille et al., “CD40- and HLA-DR-Mediated Cell Death Pathways Share a Lot of Similarities but Differ in Their Use of ADP-Ribosyltransferase Activities,” Int. Immunol. 11(5):719-730 (1999); Mimori et al., “Costimulatory Signals Distinctively Affect CD20- and B-Cell-Antigen-Receptor-Mediated Apoptosis in Burkitt's Lymphoma/Leukemia Cells,” Leukemia 17(6):1164-1174 (2003), each of which is hereby incorporated by reference in its entirety), CD30 to cysteine protease activation and caspase 8 mediated apoptosis (Fotin-Mleczek et al., “Apoptotic Crosstalk of TNF Receptors: TNF-R2-induces Depletion of TRAF2 and IAP Proteins and Accelerates TNF-R1-Dependent Activation of Caspase-8,” J. Cell Sci. 115(13):2757-2770 (2002); Tarkowski, “Expression and Function of CD30 on T Lymphocytes,” Arch. Immunol. Ther. Exp. (Warsz) 47(4):217-221 (1999), each of which is hereby incorporated by reference in its entirety), and of HLA-DR with MAP kinase activation and loss of mitochondrial membrane potential (Bains et al., “Mitochondria Control of Cell Death Induced by anti-HLA-DR Antibodies,” Leukemia 17(7):1357-1365 (2003); Drenou et al., “A Caspase Independent Pathway of MHC Class II Antigen-Mediated Apoptosis of Human B Lymphocytes,” J. Immunol. 163(8):4115-4124 (1999), each of which is hereby incorporated by reference in its entirety). Because rATG binding to many surface signaling proteins capable of activating several different apoptotic pathways was detected, examination was made as to whether any single apoptotic signaling pathway predominated (FIG. 4). CD40L activated B cells were incubated with rATG (100 μg/ml) for 18 hours in the presence or absence of apoptotic enzyme inhibitors. Significant reductions in rATG triggered apoptosis were observed with compounds inhibiting caspase activation (z-VAD-fmk), lysosomal cysteine proteases (E64d) and cathepsin B (z-FA-fmk). In contrast, inhibitors of MAP kinases (SB202474, SB203561), serine proteases (genistein), ERK (U0126), the calpains (calpain III inhibitor, PD150606) and cathepsin D (pepstatin A) had no significant effect compared with the negative control z-FK-FMK.

### Example 6

### Analysis of rATG Antibodies Against Plasma Cells

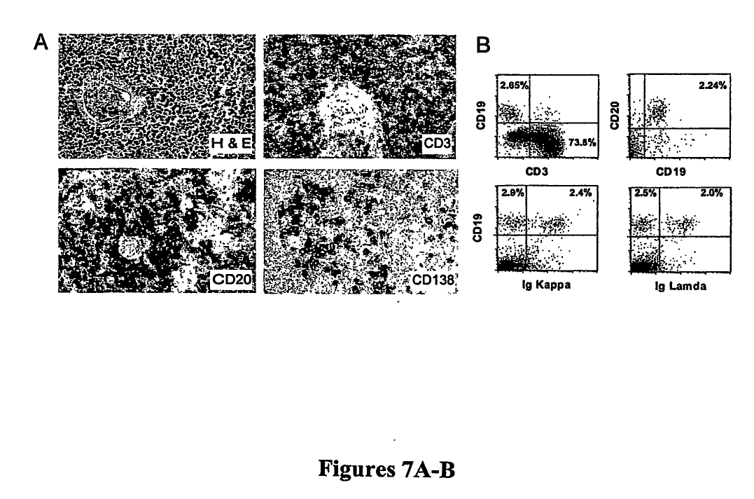

It seems counterintuitive that rATG should contain antibodies directed against B cells (e.g. CD20, HLA-DR), and especially against CD138, a plasma cell specific surface antigen. rATG is made by immunizing rabbits against cells isolated from human pediatric thymii (Bonnefoy-Berard and Revillard, “Mechanisms of Immunosuppression Induced by Antithymocyte Globulins and OKT3,” J. Heart Lung Transplant 15(5):435-442 (1996); Raefsky et al., “Biological and Immunological Characterization of ATG and ALG,” Blood 68(3):712-719 (1986), each of which is hereby incorporated by reference in its entirety). While the predominant cell type in the thymus is CD3⁺, the thymic medulla contains CD20⁺ B cells (Hofmann et al., “Thymic Medullary Cells Expressing B Lymphocyte Antigens,” Hum. Pathol. 19(11):1280-1287 (1988); Fend et al., “Phenotype and Topography of Human Thymic B cells: An Immunohistologic Study,” Arch. B Cell Pathol. Incl. Mol. Pathol. 60(6):381-388 (1991), each of which is hereby incorporated by reference in its entirety). It is believed that no prior reports exist that describe the presence of plasma cells in the human thymus. To determine if the human pediatric thymii contain mature plasma cells, immunoperoxidase staining was performed on pediatric thymii, demonstrating the presence of both CD20⁺ B cells and CD138⁺ plasma cells (FIG. 7A). Flow cytometry of Ficoll density gradient isolated thymocytes demonstrated intracellular staining for λ- and κ-light chains in approximately 6% of cells (FIG. 7B). These results indicate the presence of both B cells and plasma cells in thymocyte preparations used as an immunogen in rATG preparation.

### Example 7

### rATG Induction of Apoptosis by Multiple Myeloma Cells

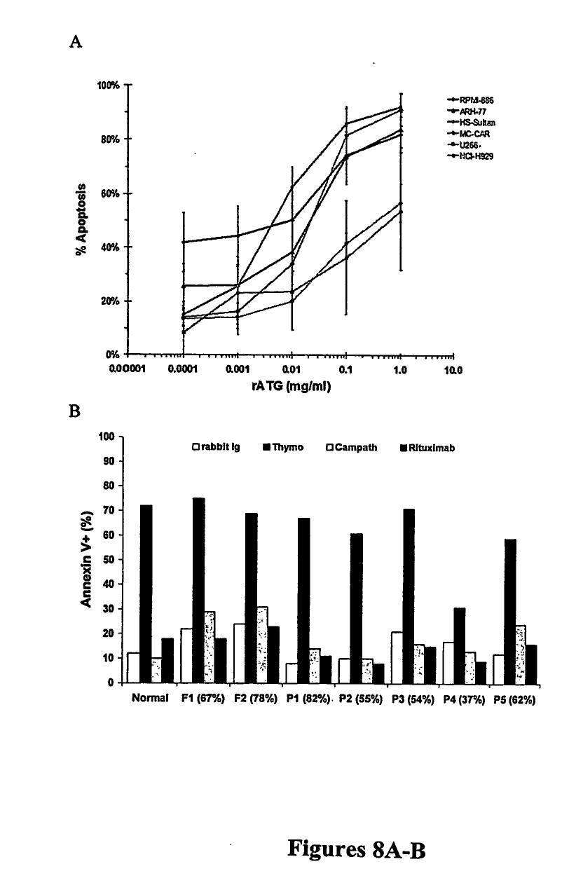

Myeloma cell lines (FIG. 8A) or bone marrow samples from multiple myeloma patients (FIG. 8B) were incubated with clinically relevant concentrations of rATG in complement free medium. Cells were assayed for apoptosis after 18 hours by flow cytometry and staining with Annexin V/TOPRO-3. rATG induced high levels of apoptosis in all myeloma cell lines, although two lines had less than 50% apoptosis at maximal concentrations. FIG. 8B is a graph illustrating the effect of rATG on bone marrow aspirates from patients with multiple myeloma, which were purified by Ficoll density gradient centrifugation and plasma/myeloma cells by CD138 affinity column positive selection. Cells were incubated with rATG 100 mcg/ml and apoptosis measured by Annexin V/TOPRO-3 staining. F=from frozen specimens, P=freshly isolated cells, (%)=percentage of marrow infiltrated with malignant cells.

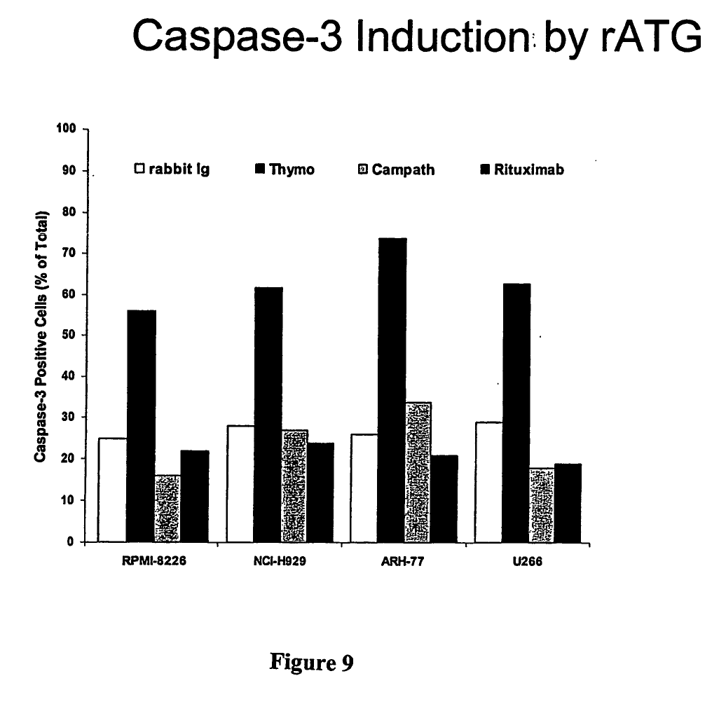

FIG. 9 is a graph illustrating the induction of caspase-3 by rATG. CD138⁺ cells selected from myeloma cell lines were incubated in 100 ng/ml rATG or rabbit IgG (control), rituxumab, or campath® (alemtuzumab) in complement free medium. rATG induced caspase-3 at substantially higher levels than control, rituxumab, or alemtuzumab.

### Discussion of Examples 1-7

Since Metchnikov first described the effects of guinea pig anti-rat splenocyte serum on rat leukocytes, the lymphotoxic effects of anti-lymphocyte sera (ALS) have been studied in detail for over a century. Clinical preparations of ALS have been made by immunizing horses or rabbits with human thymocytes or activated T or B cell blasts (Bonnefoy-Berard et al., “Antibodies Against Functional Leukocyte Surface Molecules in Polyclonal Antilymphocyte and Antithymocyte Globulins,” Transplantation 51(3):669-673 (1991); Moore, “Preparation of Antilymphocyte Globulin,” N. Engl. J. Med. 280(2):109 (1969); Najarian et al., “Anti-serum to Cultured Human Lymphoblasts: Preparation, Purification and Immunosuppressive Properties in Man,” Ann. Surg. 170(4):617-632 (1969); Monaco et al., “Some Effects of Purified Heterologous Antihuman Lymphocyte Serum in man,” Transplantation 5(4):Suppl:1106-14 (1967); Ochiai et al., Specificity and Immunosuppressive Potency of a Rabbit Antimouse T Cell-Specific Antiserum,” Transplantation 20(3):198-210 (1975), each of which is hereby incorporated by reference in its entirety) and ALS have been used to treat or prevent allograft rejection for almost four decades (Shah et al., “Treatment of CD4 Positive Acute Humoral Rejection With Plasmapheresis and Rabbit Polyclonal Antithymocyte Globulin,” Transplantation 77(9):1399-405 (2004); Monaco et al., “Some Effects of Purified Heterologous Antihuman Lymphocyte Serum in Man,” Transplantation 5(4):Suppl:1106-14 (1967); Najarian et al., “Studies of Antilymphoblast Globulin in Clinical Organ Transplantation,” Br. J. Surg. 56(8):616 (1969); Mollee et al., “Combination Therapy with Tacrolimus and Antithymocyte Globulin for the Treatment of Steroid-Resistant Acute Graft-Versus-Host Disease Developing During Cyclosporine Prophylaxis,” Br. J. Haematol. 113(1):217-223 (2001), each of which is hereby incorporated by reference in its entirety). Several studies recognized that most ALS preparations contain antibodies directed against both T and B cells (Bonnefoy-Berard et al., “Antibodies Against Functional Leukocyte Surface Molecules in Polyclonal Antilymphocyte and Antithymocyte Globulins,” Transplantation 51(3):669-673 (1991); Bonnefoy-Berard et al., “Apoptosis Induced by Polyclonal Antilymphocyte Globulins in Human B-cell Lines,” Blood 83(4):1051-1059 (1994); Raefsky et al., “Biological and Immunological Characterization of ATG and ALG,” Blood 68(3):712-719 (1986); Ochiai et al., “Specificity and Immunosuppressive Potency of a Rabbit Antimouse T Cell-Specific Antiserum,” Transplantation 20(3):198-210 (1975), each of which is hereby incorporated by reference in its entirety). However, the use of “thymocytes” as the immunogen, and the spectacular success of ALS in preventing and treating acute cellular rejection, and has led most clinicians to think of anti-thymocyte globulins as selective anti-T cell agents. In contrast, it was demonstrated that rATG prepared by immunization with pediatric human thymocytes has specific activity against surface proteins expressed by naïve and activated B cells, as well as antibody secreting plasma cells. These antibodies are a direct result of the presence of CD20⁺ B cells and CD138⁺ plasma cells in the thymocyte innocula Although equine anti-human thymocyte globulin was not tested, it seems likely that it would contain similar activity. ALS produced with purified and activated CD3⁺ lymphoblasts cells might also induce apoptosis in B cells by virtue of antibodies against shared epitopes (e.g. CD27, HLA-A/B/C, CD95, etc.), but would likely lack plasma cell activity. Similarly, ALS made using transformed B cell lines or B cell blasts would be expected to have cross-reactivity against T cells, if only via antibodies directed against MHC Class I molecules.

Although rATG can induce complement mediated lysis of lymphocytes, rapid induction of large-scale B lymphocyte apoptosis by rATG was demonstrated in B cells cultured in complement inactivated medium. All our experiments were conducted in the presence of heat treated sera which had been tested to confirm complement inactivation. However, induction of apoptosis by F(ab)₂ fragments of rATG confirms that this is a complement independent phenomenon, as F(ab)₂ preparations of rabbit IgG are incapable of binding complement. While the above examples focused on apoptotic pathways by performing experiments in complement inactivated media, the presence of complement would cause B cell necrosis in addition to apoptosis. Large scale B cell necrosis in vivo may have as yet unknown immunomodulating effects, and thus the use of F(ab)₂ fragmented rATG will require additional study to determine if it has advantages over the unmodified preparation.

It is interesting to note that binding of rATG to the FcR appears to increase the efficacy of rATG induced apoptosis. This appears to be a consequence of FcR crosslinking (for CD16 and CD32) and FcR ligation (for CD64), as this activity can be restored when F(ab)₂ fragments of rATG are combined with either divalent antibodies that crosslink, or monovalent Fc fragments which bind to, the Fcγ receptor. Ligation of the FcR in B cells under certain conditions has been reported to induce apoptosis (Ashman et al., “Fc Receptor Off-Signal in the B Cell Involves Apoptosis,” J. Immunol. 157(1):5-11 (1996), which is hereby incorporated by reference in its entirety), and FcR heterogeneity may also explain the differential sensitivity of some lupus patients to treatment with anti-CD20 (Anolik et al., “The Relationship of FcgammaRIIIa Genotype to Degree of B Cell Depletion by Rituximab in the treatment of Systemic Lupus Erythematosus,” Arthritis Rheum. 48(2):455-459 (2003), which is hereby incorporated by reference in its entirety). Thus, particularly for B cells which express high levels of FcγIII, it seems advantageous to have antibodies with a functional Fc region for both complement binding and for apoptosis induction. Many of the B cell specific markers that rATG reacts with have known roles in apoptosis signaling. Of these, it is interesting to note that only the anti-HLA-DR antibodies induced a level of apoptosis similar to that seen with rATG. Anti-HLA-DR antibodies are known to cause apoptosis of activated and naïve B cells by a caspase independent pathway (Blancheteau et al., “HLA Class II Signals Sensitize B Lymphocytes to Apoptosis Via Fas/CD95 by Increasing FADD Recruitment to Activated Fas and Activation of Caspases,” Hum. Immunol. 63(5):375-383 (2002); Bains et al., “Mitochondria Control of Cell Death Induced by anti-HLA-DR Antibodies,” Leukemia 17(7):1357-1365 (2003); Bertho et al., “HLA-DR Mediated Cell Death Is Associated With, But Not Induced by TNF-alpha Secretion in APC,” Hum. Immunol. 62(2):106-112 (2001), each of which is hereby incorporated by reference in its entirety). MHC Class II peptides are down regulated on plasma cells, however, and thus this mechanism cannot explain rATG triggered plasma cell apoptosis. Nevertheless, anti-HLA-DR monoclonal antibodies (Nagy et al., “Fully Human, HLA-DR-specific Monoclonal Antibodies Efficiently Induce Programmed Death of Malignant Lymphoid Cells,” Nat. Med. 8(8):801-807 (2002), which is hereby incorporated by reference in its entirety) may be useful in treating antibody mediated renal allograft rejection. The data reported here further suggest that one potential approach to developing new antibody therapies would be to create “poly-monoclonal” reagents: defined mixtures of monoclonal antibodies that target multiple surface proteins expressed at different stages of lymphocyte development The results reported above may also explain why two other induction agents used in renal transplantation have had limited efficacy in highly sensitized recipients. Activated B cells and bone marrow resident plasma cells are the source of alloantibodies in sensitized patients. The pan-B cell marker CD20 is down-regulated on activated B cells, and not expressed on mature bone marrow resident plasma cells. This likely explains why rituximab (anti-CD20) treatment of cynomolgus monkeys had no effect on alloantibody levels or plasma cell populations (Schroder et al., “Anti-CD20 Treatment Depletes B-cells in Blood and Lymphatic Tissue of Cynomolgus Monkeys,” Transpl. Immunol. 12(1):19-28 (2003), which is hereby incorporated by reference in its entirety). Rituximab therapy alone only modestly reduced panel reactive antibody levels in allosensitized patients on the renal transplant waiting list (Vieira et al., “Rituximab for Reduction of anti-HLA Antibodies in Patients Awaiting Renal Transplantation: Safety, Pharmacodynamics, and Pharmacokinetics,” Transplantation 77(4):542-548 (2004), which is hereby incorporated by reference in its entirety). Anti-CD52 (alemtuzumab) has also been used as induction therapy in renal transplantation (Kirk et al., “Results from a Human Renal Allograft Tolerance Trial Evaluating the Humanized CD52-specific Monoclonal Antibody Alemtuzumab (CAMPATH-1H),” Transplantation 76(1):120-129 (2003), which is hereby incorporated by reference in its entirety). As demonstrate, however, CD52 is downregulated on activated B cells and is absent on plasma cells. In this study, in vitro treatment of activated B cells and plasma cells with anti-CD52 did not cause significant apoptosis. These results may explain why renal transplant recipients treated with alemtuzumab induction therapy develop donor specific anti-HLA antibodies and have high rates of antibody mediated allograft rejection (Kirk et al., “Results from a Human Renal Allograft Tolerance Trial Evaluating the Humanized CD52-specific Monoclonal Antibody Alemtuzumab (CAMPATH-1H),” Transplantation 76(1):120-129 (2003); Cai et al., “Correlation Between Human Leukocyte Antigen Antibody Production and Serum Creatinine in Patients Receiving Sirolimus Monotherapy After Campath-1H Induction,” Transplantation 78(6):919-924 (2004), which is hereby incorporated by reference in its entirety). The induction of plasma cell apoptosis by rATG in vitro suggests that it's use as induction therapy in highly sensitized renal transplant recipients may be a successful strategy to prevent activation of alloreactive B and T cells, while reducing or eliminating alloreactive plasma cells. One potential issue with this strategy is that the relative concentrations of B versus T cell directed antibodies in the rATG preparations have not been determined. It is therefore possible that clinical anti-B cell activity of rATG may be inadequate to “de-bulk” the pre-plasma cell compartment of patients with a high frequency of alloreactive memory B cells. In such cases, it may be advantageous to use combination therapy with rATG and rituximab (anti-CD20 monoclonal antibody) or a poly(monoclonal) therapy to further boost the anti-B cell effect. Obviously, the development of targeted antiplasma cell therapies such as anti-CD138 maybe of even greater utility for treatment of highly sensitized patients, chronic alloantibody mediated graft rejection, or immune desensitization before ABO incompatible transplants (Post et al., “Efficacy of An Anti-CD138 Immunotoxin and Doxorubicin on Drug-Resistant and Drug-sensitive Myeloma Cells,” Int. J. Cancer 83(4):571-576 (1999), which is hereby incorporated by reference in its entirety). Demonstrating the utility of such strategies in solid organ transplantation will require rigorous prospective, randomized, multicenter clinical trials.

Based on early reports of rATG activity against human B cell lines, a treatment for acute antibody mediated renal allograft rejection using rATG and plasmspheresis was implemented (Shah et al., “Treatment of CD4 Positive Acute Humoral Rejection With Plasmapheresis and Rabbit Polyclonal Antithymocyte Globulin,” Transplantation 77(9):1399-405 (2004), which is hereby incorporated by reference in its entirety). The in vitro studies reported here provide a detailed scientific underpinning for this and other regimens that seek to prevent or treat alloantibody mediated transplant rejection. By extension, it is believed that rATG may have utility in the treatment of B cell mediated autoimmune disease, or as part of an induction chemotherapy regimen for autologous stem cell transplantation in the treatment of B cell and plasma cell malignancies.

Although the invention has been described in detail (both above and in the accompanying examples) for the purposes of illustration, it is understood that such detail is solely for that purpose, and variations can be made therein by those skilled in the art without departing from the spirit and scope of the invention which is defined by the following claims.

What is claimed:

1. A method of inducing B cell apoptosis comprising:
contacting a B cell with a polyclonal anti-thymocyte serum or at least one of a plurality of monoclonal antibodies, or effective fragments or variants thereof, that bind to B cell surface markers under conditions effective to induce apoptosis of the contacted B cell.

2. The method according to claim 1 wherein the B cell is selected from the group of immature B cells, naïve B cells, activated B cells, memory B cells, blastic B cells, and plasma B cells.

3. The method according to claim 1 wherein the B cell is a CD19⁺ peripheral blood B cell, CD40L activated B cell plasmablast, and/or normal human plasma cell.

4. The method according to claim 1 wherein said method is carried out using a polyclonal anti-thymocyte serum.

5. The method according to claim 4 wherein the polyclonal anti-thymocyte serum is from a primate or pig.

6. The method according to claim 1 wherein said method is carried out using at least one of a plurality of monoclonal antibodies or effective fragments thereof.

7. The method according to claim 6 wherein the monoclonal antibodies are humanized monoclonal antibodies or fragments thereof.

8. The method according to claim 6 wherein the plurality of monoclonal antibodies comprise two or more antibodies, or effective fragments or variants thereof, that recognize a B cell surface marker selected from the group of CD16, CD19, CD20, CD27, CD30, CD32, CD38, CD40, CD45, CD80, CD86, CD95, CD138, HLA-A, HLA-B, HLA-C, HLA-DR, HLA-DQ, HLA-DP, MHC Class I, MHC Class II, sIgG, sIgM, sIgD, sIgE, and sIgA, hyaluronic acid receptor, alpha interferon receptor, Ig Kappa-or lambda-light chain, Ig heavy chain, and TNF proteins.

9. The method according to claim 1 wherein the B cell is in vitro.

10. The method according to claim 1 wherein the B cell is in vivo.

11. A method of inducing apoptosis in myeloma cells comprising:
contacting a myeloma cell with a polyclonal anti-thymocyte serum or at least one of a plurality of monoclonal antibodies, or effective fragments or variants thereof, that bind to a myeloma cell surface marker under conditions effective to induce myeloma cell apoptosis.

12. The method according to claim 11 wherein said method is carried out using a polyclonal anti-thymocyte serum.

13. The method according to claim 12 wherein the polyclonal anti-thymocyte serum is from a primate or pig.

14. The method according to claim 11 wherein said method is carried out using at least one of a plurality of monoclonal antibodies or effective fragments or variants thereof.

15. The method according to claim 14 wherein the monoclonal antibodies are humanized monoclonal antibodies or fragments thereof.

16. The method according to claim 14 wherein the plurality of monoclonal antibodies or effective fragments thereof comprise two or more antibodies, or effective fragments or variants thereof, that recognize a myeloma cell surface marker selected from the group of CD16, CD19, CD20, CD27, CD30, CD32, CD38, CD40, CD45, CD80, CD86, CD95, CD138, HLA-A, HLA-B, HLA-C, HLA-DR, HLA-DQ, HLA-DP, MHC Class I, MHC Class IL sIgG, sIgM, sIgD, sIgE, and sIgA, hyaluronic acid receptor, alpha interferon receptor, Ig Kappa-or lambda-light chain, Ig heavy chain, and TNF proteins.

17. The method according to claim 14 wherein the myeloma cell is CD138⁺.

18. The method according to claim 11 wherein the myeloma cell is in vitro.

19. The method according to claim 11 wherein the myeloma cell is in vivo.

20. A method of treating multiple myeloma comprising:
providing either (i) a polyclonal anti-thymocyte serum or (ii) at least one of a plurality of monoclonal antibodies, or effective fragments or variants thereof, that bind to a myeloma cell surface marker; and
administering to a patient experiencing multiple myeloma an amount of (i) or (ii) that is effective to destroy myeloma cells, thereby treating the multiple myeloma condition.

21. The method according to claim 20 wherein said method is carried out using a polyclonal anti-thymocyte serum.

22. The method according to claim 21 wherein the polyclonal anti-thymocyte serum is from a primate or pig.

23. The method according to claim 20 wherein said method is carried out using at least one of a plurality of monoclonal antibodies or effective fragments or variants thereof.

24. The method according to claim 23 wherein the monoclonal antibodies are humanized monoclonal antibodies or fragments thereof.

25. The method according to claim 23 wherein the plurality of monoclonal antibodies, or effective fragments or variants thereof comprise two or more antibodies that recognize a myeloma cell surface marker selected from the group of CD16, CD19, CD20, CD27, CD30, CD32, CD38, CD40, CD45, CD80, CD86, CD95, CD138, HLA-A, HLA-B, HLA-C, HLA-DR, HLA-DQ, HLA-DP, MHC Class I, MHC Class II, sIgG, sIgM, sIgD, sIgE, and sIgA, hyaluronic acid receptor, alpha interferon receptor, Ig Kappa-or lambda-light chain, Ig heavy chain, and TNF proteins.

26. The method according to claim 20 wherein said administering is carried out orally, parenterally, subcutaneously, transdermally, intravenously, intramuscularly, intraperitoneally, by intranasal instillation, by implantation, by intracavitary or intravesical instillation, intraocularly, intraarterially, intralesionally, by application to mucous membranes.

27. The method according to claim 20 further comprising:
periodically repeating said administering.

28. A method of treating a B cell or plasma cell-related autoimmune disorder comprising:
providing either (i) a polyclonal anti-thymocyte serum or (ii) at least one of a plurality of monoclonal antibodies, or effective fragments or variants thereof, that bind to a B cell or plasma cell surface marker; and
administering to a patient experiencing a B cell or plasma cell-related autoimmune disorder an amount of (i) or (ii) that is effective to destroy B cells or plasma cells responsible for the autoimmune disorder, thereby treating the B cell or plasma cell-related autoimmune disorder.

29. The method according to claim 28 wherein said method is carried out using a polyclonal anti-thymocyte serum.

30. The method according to claim 29 wherein the polyclonal anti-thymocyte serum is from a primate or pig.

31. The method according to claim 28 wherein said method is carried out using at least one of a plurality of monoclonal antibodies or effective fragments or variants thereof.

32. The method according to claim 31 wherein the monoclonal antibodies are humanized monoclonal antibodies or fragments or variants thereof.

33. The method according to claim 31 wherein the plurality of monoclonal antibodies, or effective fragments thereof, comprise two or more antibodies that recognize a B cell or plasma cell surface marker selected from the group of CD16, CD19, CD20, CD27, CD30, CD32, CD38, CD40, CD45, CD80, CD86, CD95, CD138, HLA-A, HLA-B, HLA-C, HLA-DR, HLA-DQ, HLA-DP, MHC Class I, MHC Class II, sIgG, sIgM, sIgD, sIgE, and sIgA, hyaluronic acid receptor, alpha interferon receptor, Ig Kappa-or lambda-light chain, Ig heavy chain, and TNF proteins.

34. The method according to claim 28 wherein administering is carried out orally, parenterally, subcutaneously, transdermally, intravenously, intramuscularly, intraperitoneally, by intranasal instillation, by implantation, by intracavitary or intravesical instillation, intraocularly, intraarterially, intralesionally, by application to mucous membranes.

35. The method according to claim 28 further comprising:
periodically repeating said administering.

36. The method according to claim 28 wherein the B cell or plasma cell-related autoimmune disorder is selected from the group of: systemic lupus erythematosus, Rheumatoid arthritis, diabetis, Sjogren's syndrome, Hashimoto's disease, Wegner's granulomatosis, polyarteritis nodosum, anti-cardiolipin antibody syndrome, autoimmune hepatitis, and B cells cancers of the immune system.

37. A method of treating a patient for a B cell malignancy comprising:
providing either (i) a polyclonal anti-thymocyte serum or (ii) at least one of a plurality of monoclonal antibodies, or effective fragments or variants thereof, that bind to a malignant B cell surface marker, and
administering to a patient experiencing a B cell malignancy an amount of (i) or (ii) that is effective to destroy malignant B cells, thereby treating the patient for the B cell malignancy.

38. The method according to claim 37 wherein said method is carried out using a polyclonal anti-thymocyte serum.

39. The method according to claim 38 wherein the polyclonal anti-thymocyte serum is from a primate or pig.

40. The method according to claim 37 wherein said method is carried out using at least one of a plurality of monoclonal antibodies or effective fragments or variants thereof.

41. The method according to claim 37 wherein the monoclonal antibodies are humanized monoclonal antibodies or fragments thereof.

42. The method according to claim 37 wherein the plurality of monoclonal antibodies, or effective fragments or variants thereof, comprise two or more antibodies that recognize a myeloma cell surface marker selected from the group of CD16, CD19, CD20, CD27, CD30, CD32, CD38, CD40, CD45, CD80, CD86, CD95, CD138, HLA-A, HLA-B, HLA-C, HLA-DR, HLA-DQ, HLA-DP, MHC Class I, MHC Class II, sIgG, sIgM, sIgD, sIgE, and sIgA, hyaluronic acid receptor, alpha interferon receptor, Ig Kappa-or lambda-light chain, Ig heavy chain, and TNF proteins.

43. The method according to claim 37 wherein said administering is carried out orally, parenterally, subcutaneously, transdermally, intravenously, intramuscularly, intraperitoneally, by intranasal instillation, by implantation, by intracavitary or intravesical instillation, intraocularly, intraarterially, intralesionally, by application to mucous membranes.

44. The method according to claim 37 further comprising:
periodically repeating said administering.

45. A method of treating B cell or plasma cell-related alloantibody disorders in solid organ or bone marrow transplantation, said method comprising:
providing either (i) a polyclonal anti-thymocyte serum or (ii) at least one of a plurality of monoclonal antibodies, or effective fragments or variants thereof, that bind to a B cell or plasma cell surface marker on B cells or plasma cells that are implicated in an alloantibody disorder, and
administering to a patient experiencing a B cell or plasma cell-related autoimmune disorder an amount of (i) or (ii) that is effective to destroy B cells or plasma cells responsible for the autoimmune disorder, thereby treating the B cell or plasma cell-related alloantibody disorder.

46. The method according to claim 45 wherein said method is carried out using a polyclonal anti-thymocyte serum.

47. The method according to claim 46 wherein the polyclonal anti-thymocyte serum is from a primate or pig.

48. The method according to claim 45 wherein said method is carried out using at least one of a plurality of monoclonal antibodies or effective fragments or variants thereof.

49. The method according to claim 46 wherein the monoclonal antibodies are humanized monoclonal antibodies or fragments thereof.

50. The method according to claim 45 wherein the plurality of monoclonal antibodies, or effective fragments or variants thereof, comprise two or more antibodies that recognize a myeloma cell surface marker selected from the group of CD16, CD19, CD20, CD27, CD30, CD32, CD38, CD40, CD45, CD80, CD86, CD95, CD138, HLA-A, HLA-B, HLA-C, HLA-DR, HLA-DQ, HLA-DP, MHC Class I, MHC Class II, sIgG, sIgM, sIgD, sIgE, and sIgA, hyaluronic acid receptor, alpha interferon receptor, Ig Kappa-or lambda-light chain, Ig heavy chain, and TNF proteins.

51. The method according to claim 45 wherein said administering is carried out orally, parenterally, subcutaneously, transdermally, intravenously, intramuscularly, intraperitoneally, by intranasal instillation, by implantation, by intracavitary or intravesical instillation, intraocularly, intraarterially, intralesionally, by application to mucous membranes.

52. The method according to claim 45 further comprising:
periodically repeating said administering.

53. A composition comprising two or more monoclonal antibodies or fragments or variants thereof that are effective in binding to a B cell or plasma cell surface marker, and either individually or collectively inducing apoptosis to the bound cell.

54. The composition according to claim 53 wherein the B cell or plasma cell surface marker is selected from the group of CD16, CD19, CD20, CD27, CD30, CD32, CD38, CD40, CD45, CD80, CD86, CD95, CD138, HLA-A, HLA-B, HLA-C, HLA-DR, HLA-DQ, HLA-DP, MHC Class I, MHC Class II sIgG, sIgM, sIgD, sIgE, and sIgA, hyaluronic acid receptor, alpha interferon receptor, Ig Kappa-or lambda-light chain, Ig heavy chain, and TNF proteins.

55. The composition according to claim 53 wherein the monoclonal antibodies or fragments or variants thereof are humanized monoclonal antibodies or fragments thereof.

56. The composition according to claim 53 comprising three or more monoclonal antibodies or fragments or variants thereof.
